(19) United States
(12) Patent Application Publication
(10) Pub. No.: US 2026/0334933 A1
(43) Pub. Date: Oct. 29, 2026

(54) TISSUE TRACTION DEVICES, SYSTEMS, AND METHODS

(71) Applicant: Boston Scientific Scimed, Inc., Maple Grove, MN (US)

(72) Inventors: Scott Edward Corbeil, Litchfield, NH (US); Julie Gagnon, Providence, RI (US)

(73) Assignee: Boston Scientific Scimed, Inc., Maple Grove, MN (US)

(21) Appl. No.: 19/659,120

(22) Filed: Apr. 27, 2026

Related U.S. Application Data
(60) Provisional application No. 63/795,931, filed on Apr. 28, 2025.

(51) Int. Cl. A61B 17/02; A61B 17/00; A61B 17/064

(52) U.S. Cl. CPC A61B 17/0218; A61B 2017/00269; A61B 2017/0034; A61B 2017/00862; A61B 2017/0225; A61B 2017/0649

(57) ABSTRACT

A tissue traction system with a traction device having two or more distinct geometric zones. The distal tissue-engagement zone is in the form of a helical coil configured to securely engage or anchor the traction device with respect to target tissue. A delivery device of the system may have a delivery head engageable with the tissue traction device to rotate and advance the tissue-engagement zone into tissue. The tissue traction device may include a helical traction zone which applies traction to the distal tissue-engagement zone and into which the delivery head of the delivery device may be insertable. A final zone may include an attachment zone by which the end of the traction device opposite the tissue-engagement zone may be coupled with respect to a traction-adjusting site to apply traction to the target tissue.

### CROSS-REFERENCE TO RELATED APPLICATIONS

This application claims the benefit of priority under 35 U.S.C. § 119 to U.S. Provisional Application No. 63/795,931, filed on Apr. 28, 2025, the entire disclosure of which is hereby incorporated by reference herein for all purposes.

### FIELD

The present disclosure relates generally to the field of devices, systems, and methods for applying traction to anatomical tissue.

### BACKGROUND

Endoscopic Mucosal Resection (EMR) is an endoscopic procedure which involves the placement of a snare around tissue and either contracting the snare to transect (cold), or using electrosurgical current to transect (hot) the encircled tissue. Endoscopic Submucosal Dissection (ESD) is an endoscopic procedure, first developed in Japan in the late 1990's, used to remove superficial gastrointestinal neoplasms via en bloc resection as a reaction to the shortcomings of EMR. Typically, ESD procedures are technically more challenging than EMR procedures, with longer procedural times and higher rates of complications. However, en bloc resection allows good histopathological assessment of lesion margins, lower recurrence rates, and a greater minimally invasive potential for cure.

Minimally-invasive (e.g., transluminal, transcatheter, endoscopic, etc.) surgical techniques like ESD and EMR typically allow for faster recovery than with open or laparoscopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the instruments being used, occluding visibility of the imaging device (e.g., camera), and creating a hindrance affecting movement of the instruments used during the procedure and in reaching all regions and depths of the target tissue being cut. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Moreover, as tissue is cut, a continual increase in traction is required, presenting various challenges during the procedure. Additionally, the devices used with such solutions may require separate medical tools than those used to perform the procedure, and such tools may even require a separate working channel, thereby potentially increasing the size and/or complexity of the delivery system. As procedures such as endoscopic submucosal dissection continue to evolve with new techniques, and new instruments are designed for making the procedure faster and safer, there remains a need for improvements to application of traction to tissue during the ESD procedure. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a traction device for applying traction to anatomical tissue within a patient is formed in accordance with various principles of the present disclosure with a traction zone configured to impart traction to the anatomical tissue; and a tissue-engagement zone operably coupled with the traction zone. In some aspects, the tissue-engagement zone is in the form of a helical coil.

In some aspects, the traction zone is in the form of a helical coil spring. In some aspects, the traction zone and the tissue-engagement zone are formed from the same monolithic elongate element. In some aspects, the traction zone and the tissue-engagement zone are formed separately from each other and are coupled together via a coupler.

In some aspects, the distalmost end of the tissue-engagement zone is sharp and pointed to penetrate target tissue to which the traction device is to apply traction.

In some aspects, the traction device further includes comprising an additional zone operably coupled to the traction zone and configured to be attached to tissue spaced apart from target tissue to which the tissue-engagement zone is to be attached. In some aspects, the additional zone is configured to be grasped by a tissue-engagement element configured to grasp the additional zone and the tissue spaced apart from the target tissue. In some aspects, the additional zone is in the form of a helical coil.

In some aspects, a tissue traction system for applying traction to anatomical tissue within a patient, in accordance with various principles of the present disclosure, includes a tissue traction device defining a tissue-engagement zone in the form of a helical coil, and a traction zone extending proximally from the tissue-engagement zone; and a delivery device having a delivery head configured to engage the tissue traction device and to rotate the delivery device to rotationally advance the tissue-engagement zone into anatomical tissue.

In some aspects, the tissue traction device defines a cross-bar extending across the cross-section of the tissue traction device and configured to be rotationally engaged by the delivery head of the delivery device. In some aspects, the delivery head includes a pair of jaws positionable with respect to the tissue traction device to grasp the cross-bar of the tissue traction device. In some aspects, the delivery head defines a slot for receiving the cross-bar of the tissue traction device.

In some aspects, the tissue traction device includes a coupler joining the tissue-engagement zone and the traction zone of the, the coupler configured to be rotationally engaged by the delivery head of the delivery device. In some aspects, the delivery head includes a pair of jaws movable towards each other to grasp a portion of the coupler to impart rotational movement to the tissue traction device. In some aspects, the delivery head defines a rotational engagement structure configured to engage a rotational engagement structure of the coupler to impart rotational movement to the tissue traction device.

In some aspects, the delivery head includes a pair of jaws movable towards each other to grasp a portion of the tissue traction device to impart rotational movement to the tissue traction device. In some aspects, the tissue traction device further includes an additional zone extending proximally from the traction zone. In some aspects, the jaws are positionable within the traction zone to deliver the tissue traction device to target tissue within the patient, and are removable from within the traction zone to grasp the additional zone and anatomical tissue.

In some aspects, a method of applying traction to anatomical tissue in accordance with various principles of the present disclosure includes engaging a distal end of a helical tissue-engagement zone of a tissue traction device with target tissue along the anatomical tissue; rotating the helical tissue-engagement zone to advance the tissue-engagement zone into the anatomical tissue; and coupling a traction zone of the tissue traction device, proximally extending from the helical tissue-engagement zone, to anatomical tissue at a traction-adjusting site spaced apart from the target tissue to apply traction to the target tissue.

In some aspects, the traction zone is in the form of a helical coil, and rotating the helical tissue-engagement zone includes rotating a delivery head of a delivery device within the helical traction zone to cause the helical tissue-engagement zone to rotate.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tissue traction system in accordance with aspects of the present disclosure.

FIG. 2 illustrates a perspective view of a tissue traction device and delivery device such as illustrated in FIG. 1.

FIG. 3 illustrates a tissue traction device as in FIG. 2, without the tissue traction device.

FIG. 4 illustrates another example of an embodiment of a tissue traction device formed in accordance with various principles of the present disclosure.

FIG. 5A, FIG. 5B, and FIG. 5C illustrate an example of use of a tissue traction system such as illustrated in FIG. 1.

FIG. 6 illustrates another example of an embodiment of a tissue traction system formed in accordance with various principles of the present disclosure.

FIG. 7 illustrates another example of an embodiment of a tissue traction system formed in accordance with various principles of the present disclosure.

FIG. 8A, FIG. 8B, and FIG. 8C illustrate another example of an embodiment of a tissue traction system formed in accordance with various principles of the present disclosure.

FIG. 9A and FIG. 9B illustrate another example of an embodiment of a tissue traction system formed in accordance with various principles of the present disclosure.

FIG. 10 illustrates an alternate coupler which may be used in a tissue traction system such as illustrated in FIG. 9A and FIG. 9B.

FIG. 11 illustrates another example of an embodiment of a tissue traction system formed in accordance with various principles of the present disclosure.

FIG. 12 illustrates another example of an embodiment of a tissue traction system formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

There has been a growing interest in the medical field in minimally-invasive procedures, e.g., transcatheter, endoscopic, laparoscopic, etc., which do not require open surgery (cutting open the patient), but, instead, access a target site within the patient via a natural orifice (or, in some instances, a small incision not considered to constitute an open-surgery cut). For the sake of convenience, and without intent to limit, such procedures may be broadly referenced herein as transluminal procedures, in contrast with surgical procedures utilizing large surgical openings made in the patient for generally direct access (e.g., without navigating through a device or internal body passage) to the site at which a procedure is to be performed (e.g., target tissue). Various endoluminal surgical (ELS) procedures have been developed, such as, without limitation, Endoscopic Mucosal Resection (EMR), Endoscopic Submucosal Dissection (ESD), to be performed within a patient and not in direct view of the medical professionals performing the procedure. The lack of direct visibility, as well as typical limited space for maneuvering of medical instruments presents various challenges to the medical professionals performing the procedure.

Although EMR can be performed in piecemeal technique, pathology on tissue fragments is often challenging and patients require more frequent follow-up endoscopies due to higher rate of incomplete resections and recurrences. While EMR is associated with relatively low technical complexity, shorter procedure times, and a low risk of adverse events, en bloc EMR is limited typically to lesions which are less than 2.0 cm in diameter/width. More specifically, for large sessile or laterally spreading polyps, en bloc resection with convention EMR is nearly impossible for lesions greater than 2 cm. For colorectal lesions of greater than 2 cm, en bloc resection can only be achieved in about 30% of cases.

Medical professionals thus often turn to ESD for resection of challenging lesions, as well as large lesions. It is not uncommon for ESD to be utilized to remove neoplasms between 2-7 cm. The European Society of Gastrointestinal Endoscopy (ESGE) recommends ESD be used first line in the removal of Gastric Superficial neoplastic lesions with very low risk of lymph node metastases (EMR may be considered if the lesion is less than 10 to 15 mm with a very low probability of advanced histology). Often, ESD is also recommended first line for superficial esophageal squamous cell cancer (with EMR an option for lesions which are less than 10 mm). For colonic and rectal lesions, ESGE recommends removal of lesions with polypectomy or EMR. However, ESD can be considered if there is a high suspicion of limited submucosal invasion (indicated by depressed morphology and an irregular or non-granular surface pattern), and especially if the lesion is greater than 20 mm, or in lesions that otherwise cannot be optimally and radically removed by snare-based techniques (such as EMR techniques).

Although ESD has been in practice for two decades, ESD remains a very technically challenging procedure. In Japan, where ESD procedure is best established, the complication rates (including perforation, peritonitis, and bleeding) are 3.5% for gastric ESD, 3.3% for esophageal ESD, and 4.6% for Colorectal ESD. Outside of Japan and China, EDS is practiced by a relatively small number of highly technically-skilled endoscopists and surgeons. Long procedure times and high risk of perforation are still significant challenges which have prevented broad physician adoption.

In accordance with various principles of the present disclosure, devices, systems, and methods are described which may be used in minimally-invasive procedures, such as ESD, to move anatomical tissue (typically a flap of tissue formed by cutting a target tissue with respect to adjacent or surrounding tissue at a target site) out of the way of instruments being used to perform a procedure within the patient's body. It will be appreciated that reference may be made to a target site, treatment site, anatomical site, delivery site, deployment site, implant/implantation site, site of implantation, etc., interchangeably and without intent to limit. In some aspects, target tissue is defined within the treatment site and with respect to which a procedure is to be performed. The target tissue may be, but is not necessarily limited to, a lesion. It will be appreciated that reference may be made herein simply to “tissue” instead of “anatomical tissue” or “anatomical structure” for the sake of convenience and without intent to limit. In some aspects, the present disclosure is directed to devices, systems, and methods for moving tissue, such as to separate and/or lift and/or apply traction to tissue within a patient's body. Traction is a foundational technique utilized in open and laparoscopic surgery. Traction may be used to move tissue or other anatomical structures and may be advantageously used to greatly enhance visualization of anatomy, tissue planes, and potential hazards such as nerves or blood vessels. Additionally or alternatively, traction may be used to impart a tension force to the tissue, which may change the way the tissue behaves as it is being cut or dissected. For instance, traction may be used in surgical procedures to “present” the tissue to be cut to the cutting instrument in an orientation or manner that makes the cutting easier and/or helps to ensure that the cut is made through the desired tissue in the desired direction. In some aspects, traction alters the nature and/or properties of tissue in a manner which may facilitate performance of a procedure with respect to or in the vicinity of the tissue under traction. It will be appreciated that principles of the present disclosure are applicable to tissue which is cut as well as tissue or other anatomical structures which are not cut. For instance, principles of the present disclosure are applicable for providing traction and/or counter-traction during various medical procedures, including, without limitation, surgical procedures that involve cutting of tissue. The use of traction is a basic precept of surgery during cutting procedures, but should be appreciated as having broader applicability as well.

In addition to the procedural benefits of using traction during endoscopic procedures, there is a mounting body of evidence that devices aiding with traction during endoscopic submucosal dissection reduce the technical complexity of the procedure, and significantly lower the learning curve associated with trainees and/or new ESD procedure adopters.

To date, current traction solutions, both commercialized and physician made, do not adequately address true user needs. At best, commercially-available traction solutions leave significant room for improvement with regard to efficiency, effectiveness, flexibility, and ease of use. In open surgery, traction typically is applied using the non-dominant hand directly or through a surgical instrument (typically some kind of forceps). In laparoscopic surgery, traction is applied via tissue graspers introduced through one of the surgical ports, which provide access to the target body cavity or space. This can be done by the lead surgeon, assisting surgeon, or scrub nurse. In endoscopic surgery, however, multiple portals for accessing the target surgical site generally are not provided, and, typically, only the endoscope (or other access device) provides both visualization and access. Although there are some endoscopes with two working channels, or devices that can attach to the outside of the endoscope to provide a secondary working channel, all of these access channels are either integral to the endoscope or run parallel to it, and therefore are directionally constrained, and limited in the degrees of freedom of movement and control that can be achieved when maneuvering other devices to the target site. Moreover, the medical scopes and even auxiliary lumens, and often also the anatomical site of the procedure itself, offer limited space for manipulation of additional instruments, tools, devices, accessories, etc. (such terms being usable interchangeably herein without intent to limit), such as to manipulate and move tissue out of the way of the instrument performing a procedure with respect to the tissue.

In accordance with various principles of the present disclosure, a tissue traction device is formed with more than one distinct geometric zone, each zone sized, shaped, configured, dimensioned, and/or relatively positioned for use in a different manner in applying traction to tissue. The tissue traction device includes a traction zone configured, shaped, formed, etc., to impart traction or tension force to anatomical tissue. The traction zone of the tissue traction device may be flexible and/or elastic or otherwise formed to be capable of applying traction to tissue. The traction zone of the tissue traction device may be stretchable/elongatable, or non-elongatable. In some aspects, the traction zone is in the form of a helical coil.

In some aspects, a tissue traction device formed in accordance with various principles of the present disclosure includes a distal zone sized, shaped, configured, and/or dimensioned for engagement with and/or anchoring with respect to tissue. In some aspects, the distal zone is helical or coil or spiral or similarly shaped to be engaged with tissue and rotated to be advanced into secure engagement with the tissue for anchoring the tissue traction device with respect to the tissue. In some aspects, the distal zone includes a tip capable of penetrating or piercing tissue. Additionally or alternatively, a small opening may be formed by another instrument (e.g., a cutting instrument) to facilitate initial insertion of the tissue traction device into the tissue.

In some aspects, the tissue traction device of the present disclosure includes an attachment zone. In some aspects, the attachment zone extends from an end of the traction zone opposite the end from which the tissue-engagement zone extends. The attachment zone is sized, shaped, configured, dimensioned, and/or positioned for engagement with and/or anchoring with respect to tissue. In some aspects, the attachment zone is in the form of an attachment element capable of being grasped by a separate tissue-engagement element which engages tissue at a traction-adjusting location spaced apart from the target tissue to couple the attachment zone of the tissue traction device with tissue along the traction-adjusting location. For instance, in some aspects, the attachment zone is in the form of an attachment element is in the form of a loop engageable by a grasper/tissue-engagement element such as a jaw of a pair of jaws of a tissue clip.

It will be appreciated that terms such as engage (and other grammatical forms thereof) may be used interchangeably herein with terms such as affix, anchor, associate, attach, clip, clamp, clasp, couple, deploy on, grasp, hold, join, secure, etc. (and other grammatical forms thereof), without intent to limit, unless otherwise stated. Such engagement may be direct or indirect.

As may be appreciated, in order to impart rotational movement of a delivery device of a tissue traction system formed in accordance with various principles of the present disclosure to a tissue traction device formed in accordance with various principles of the present disclosure, it may be desirable for the tissue traction device and the delivery device to be operably rotationally coupled in a non-rotatable manner with respect to each other so that rotation of the delivery device causes rotation of the tissue traction device. One or both of the tissue traction device or the delivery device may be configured to facilitate rotational movement of the delivery device to be transmitted to rotational movement of the tissue traction device.

In accordance with various principles of the present disclosure, the tissue traction device is a unitary element with the different zones operably coupled together to form a unitary tissue traction device. In some aspects, the different zones are formed from the same elongate element which is shaped in different manners to differentiate the zones from one another. In some aspects, the attachment zone is formed from a proximal end of the traction zone of the tissue traction device. In some aspects, the different zones are formed by one or more different components which are separate elements operably associated with one another to form a unitary traction device. In some aspects, the elongate element forming the zones of the traction device is an elongate wire. However, other elements, and/or additional components may be used to form a tissue traction device in accordance with various principles of the present disclosure.

Various embodiments of tissue traction devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

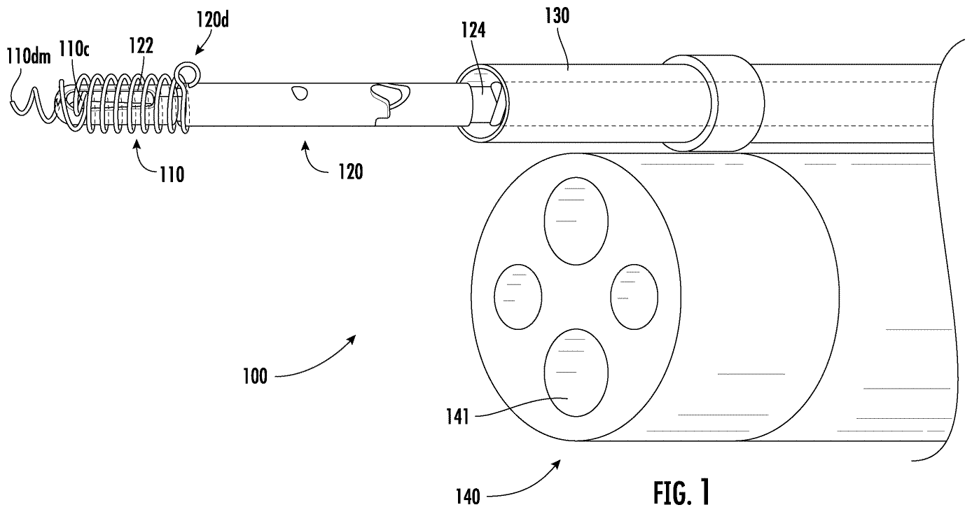

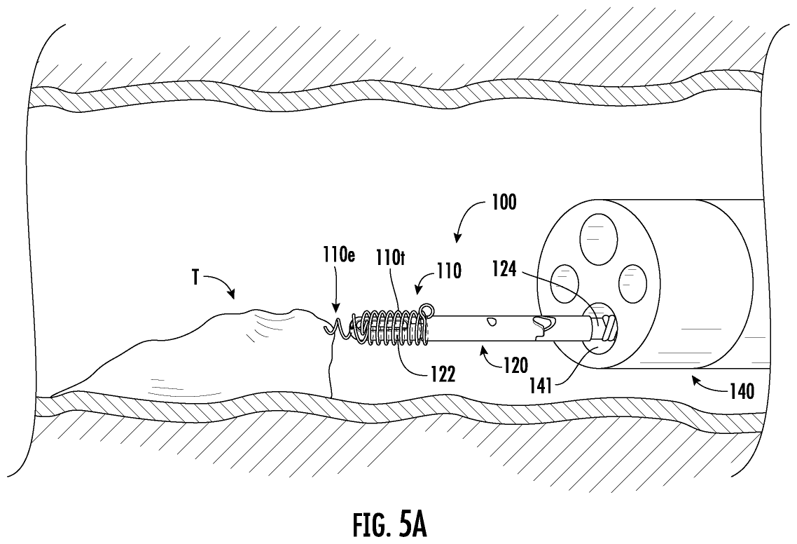

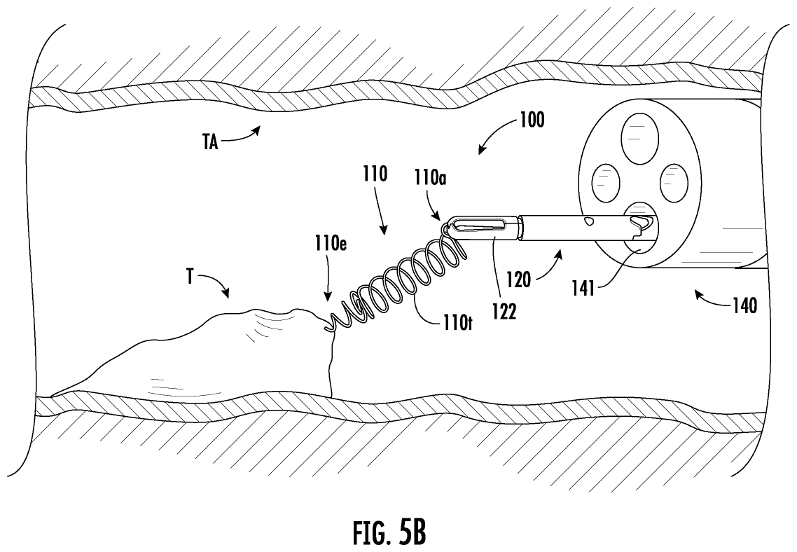

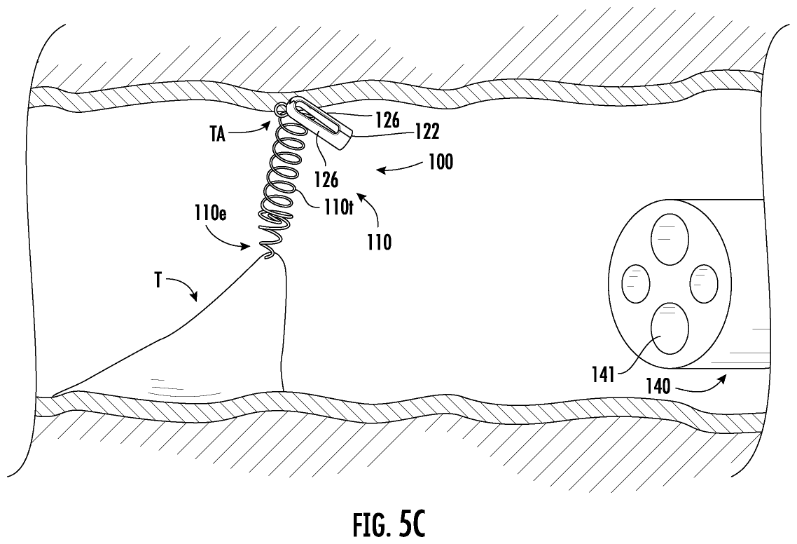

Turning now to the drawings, an example of an embodiment of a tissue traction system 100 formed in accordance with various principles of the present disclosure is illustrated in FIG. 1. The tissue traction system 100 includes a tissue traction device 110, and a delivery device 120 with a distal end 120d configured to deliver the tissue traction device 110 to target tissue (such as illustrated in FIG. 5A and FIG. 5B and discussed in further detail below). More particularly, In the illustrated example of an embodiment, the tissue traction device 110 is delivered over a delivery head 122 at (extending from or formed from) the distal end 120d of the delivery device 120. In some aspects, a delivery head described herein may be considered a driver, drive head, delivery catheter, etc., which engages the tissue traction device 110 to advance (e.g., axially and/or rotationally) the tissue traction device 110 into engagement with tissue (such as to anchor, fix, secure, etc., the tissue traction device 110 with respect to tissue). As such, the delivery head 122 may be operably coupled with the tissue traction device 110 so that movement (axial, rotational, etc.) of the delivery head 122 effects movement (axial, rotational, etc.) of the tissue traction device 110 (e.g., to drive the tissue traction device 110 into tissue). In some aspects, the delivery head 122 extends distally from or over a distal end 124d of a flexible elongate member 124 which extends proximally for control by a medical professional (e.g., to control advancement, delivery, deployment, etc., of the tissue traction system 100, or at least the tissue traction device 110 and delivery device 120 thereof). In some aspects, the delivery head 122 of the delivery device 120 is a grasper device, as illustrated in FIG. 2, configured to grasp an additional element or component such as known to those of ordinary skill in the art. In some aspects, a delivery head 122 in the form of a grasper device is, more particularly, in the form of tissue-engagement element or clip with at least first and second jaws 126 movable with respect to each other (towards/away from each other), discussed in further detail below. It will be appreciated that other forms of graspers known to those of ordinary skill in the art, such as hooks, magnetic elements, elements configured to achieve an interference-fit with another element, elements configured to achieve a lock and key type engagement with another element, etc., are within the scope of the present disclosure. In some aspects, the flexible elongate member 124 is sufficiently flexible to be extended (e.g., through a natural opening or orifice, or through a small incision) into a curved and/or tortuous passage within a patient's body and advanced (e.g., transluminally) to a target tissue within the patient's body. In some aspects, the flexible elongate member 124 is a torque wire or guide wire such as known to those of ordinary skill in the art for use in guiding and/or delivering a medical device or component into a patient's body.

In some aspects, the tissue traction device 110 and the delivery device 120 are delivered through a delivery sheath 130, which optionally may be considered a part of the tissue traction system 100. In the example of an embodiment of a tissue traction system 100 illustrated in FIG. 1, the delivery sheath 130 is extended external to and alongside the exterior of a medical delivery device 140 (such as a medical scope). Additionally or alternatively, the delivery sheath 130 may be delivered through a working channel 141 of a medical delivery device 140 (e.g., a working channel 141 of a medical delivery device 140 in the form of a medical scope, as illustrated in FIG. 5A and FIG. 5B and discussed in further detail below).

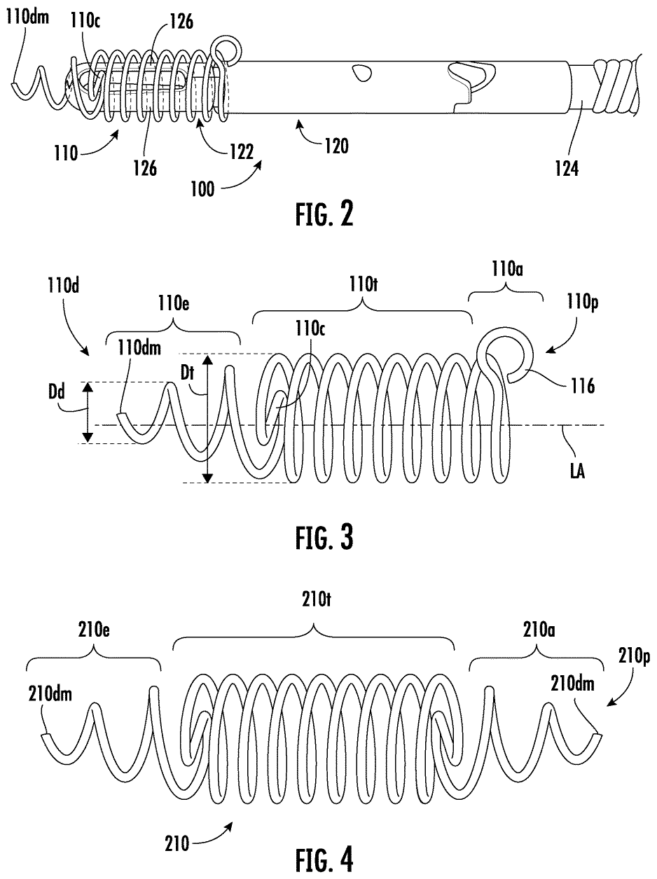

The example of an embodiment of a tissue traction device 110 illustrated in FIG. 1 is illustrated separate from the delivery device 120 in FIG. 3 to more readily appreciate various aspects of a tissue traction device 110 formed in accordance with various principles of the present disclosure. In accordance with various principles of the present disclosure, the tissue traction device 110 is differentiated along its length (along a longitudinal axis LA) into two or more regions or zones. As may be appreciated with reference to FIG. 3, in some aspects, the tissue traction device 110 is formed of a wire-form element which is shaped to form the various regions or zones along the length of the tissue traction device 110. However, a tissue traction device formed in accordance with various principles of the present disclosure need not be limited in this regard in view of other separate and independent aspects of the present disclosure which are not necessarily limited to a wire-form constructions of the tissue traction device 110. The example of an embodiment of a tissue traction device 110 illustrated in FIGS. 1-3 is configured to be able to impart traction on tissue with which the tissue traction device 110 is engaged, and thus defines a traction zone 110t configured to impart traction to tissue engaged by the distal end 110d of the tissue traction device 110. In the illustrated example of an embodiment, the traction zone 110t is in the form of a helical coil spring. However, it will be appreciated that the traction zone 110t may have other configurations without detracting from the principles of the present disclosure. The tissue traction device may be formed from a variety of materials, including, without limitation,

The distal tissue-engagement zone 110e of the tissue traction device 110 extends distally from the traction zone 110t (generally along a distal end 110d of the tissue traction device 110), as may be appreciated with reference to FIG. 3. In accordance with various principles of the present disclosure, the distal tissue-engagement zone 110e is in the form of a helical coil (cylindrical or conical), such as a helical coil spring, which may be rotated to be distally advanced into tissue. In some aspects, the helical shape facilitates advancement of the distal tissue-engagement zone 110e by relying more so on the geometry of the distal tissue-engagement zone 110e than on distal force, thereby reducing the amount of distal force required to deploy and securely engage (e.g., anchor) the tissue traction device 110 with respect to tissue. Such configuration may have various benefits, such as ease of use, increased control of distal advancement, reduced risk of puncture, etc., as may be appreciated by those of ordinary skill in the art. In some aspects, the distalmost end 110dm of the distal tissue-engagement zone 110e is sized, shaped, configured, and/or dimensioned to facilitate penetration of the distal tissue-engagement zone 110e into tissue upon engagement with tissue. For instance, the distalmost end 110dm may be pointed or sharp to penetrate or pierce the tissue sufficiently to facilitate the desired advancement of the distal tissue-engagement zone 110e into tissue without requiring a degree of force which may risk uncontrolled distal advancement (e.g., excessive puncture or damage) of the distal tissue-engagement zone 110e. In some aspects, a tissue traction device 110 formed of a wire-form element is coiled at a first diameter Dₜ to form the traction zone 110t, and at a second diameter D_{d} to form the distal tissue-engagement zone 110e. In some aspects, the change in the diameters Dₜ and D_{d} between the traction zone 110t and the distal tissue-engagement zone 110e may form an insertion stop for the tissue traction device 110. In some aspects, the distal tissue-engagement zone 110e is in the form of a conical helical coil, with the second diameter D_{d} varying along the length of the distal tissue-engagement zone 110e. It will be appreciated that the greatest diameter D_{d} of the conical helical coil is smaller than the diameter Dₜ of the traction zone 110t.

As may be appreciated, a tissue traction device 110 formed in accordance with various principles of the present disclosure may be used to apply traction to a target tissue, to which a distal end 110d of the tissue traction device 110 is engaged, by engaging a proximal end 110p of the tissue traction device 110 with tissue at a traction-adjusting site within the patient's body. The traction-adjusting site typically is tissue spaced apart from the target tissue. In use within a body lumen, the traction-adjusting site may be a wall of the body lumen across (e.g., substantially diametrically opposite or at least circumferentially spaced apart) from the target tissue, although other anatomical sites for use of a tissue traction device 110 of the present disclosure are within the scope of the present disclosure. In view of such use, in some aspects, the proximal end 110p of a tissue traction device 110 formed in accordance with various principles of the present disclosure is configured to be engaged with tissue typically while the distal end 110d is also engaged with tissue (at a separate location within the patient's body). For instance, in some aspects, the proximal end 110p of the tissue traction device 110 defines an additional zone 110a of the tissue traction device 110 (extending from an end of the traction zone 110t opposite the end from which the distal tissue-engagement zone 110e extends) configured for grasping by a medical instrument configured to manipulate the proximal end 110p of the tissue traction device 110 and/or to engage the proximal end 110p of the tissue traction device 110 with tissue at a traction-adjusting site on the patient's body. In some aspects, the additional zone 110a is in the form of an attachment member, such as a loop, hook, snare, etc., configured to be grasped by another element and/or configured to facilitate attachment of another element thereto. In some aspects, the other element is a grasper device (e.g., a grasper device as described above, such as forming the delivery head 122 of the delivery device 120). For instance, in some aspects, a grasper such as a tissue-engagement element (e.g., a clip) may be attached to the additional zone 110a as well as to tissue (e.g., tissue at a tissue-adjusting site) to engage the proximal end 110p of the tissue traction device 110 with tissue to apply traction to tissue engaged by the distal end 110d of the tissue traction device 110.

In some aspects, the proximal end of a tissue traction device formed in accordance with various principles of the present disclosure is itself configured to engage tissue. For instance, in the example of an embodiment of a tissue traction device 210 illustrated in FIG. 4, an additional zone 210a formed at the proximal end 210p of the tissue traction device 210 is similar to the above-described distal tissue-engagement zone 110e and capable of engaging tissue without the need for assistance by an additional tissue-engagement element (e.g., directly engage tissue to be secured with respect to the tissue). For instance, one or both of the distalmost end 210dm of the distal tissue-engagement zone 210e and the proximalmost end 210pm of the additional zone 210a of the example of an embodiment of a tissue traction device 210 illustrated in FIG. 4 may be sized, shaped, configured, and/or dimensioned to facilitate penetration thereof into tissue upon engagement with tissue. For instance, one or both of the distalmost end 210dm and the proximalmost end 210pm of the tissue traction device 210 may be pointed or sharp to penetrate or pierce the tissue sufficiently to facilitate the desired advancement of the respective distal tissue-engagement zone 210e or additional zone 210a into tissue without requiring a degree of force which may risk uncontrolled distal advancement (e.g., excessive puncture or damage) of the distal tissue-engagement zone 210e and/or the additional zone 210a. It will be appreciated that various features of the distal tissue-engagement zone 210e described above are applicable, mutatis mutandis, to the distal tissue-engagement zone 210e and/or the additional zone 210a of the tissue traction device 210 illustrated in FIG. 4, reference being made to the above descriptions for the sake of brevity and without intent to limit.

A tissue traction system formed in accordance with various principles of the present disclosure may be used as illustrated in FIG. 5A, FIG. 5B, and FIG. 5C. As illustrated schematically in FIG. 5A, the tissue traction system 100 may be advanced to target tissue T through a working channel 141 of a medical scope 140. The distal tissue-engagement zone 110e of the illustrated tissue traction device 110 may be engaged securely with the target tissue T so that traction forces applied to the tissue traction device 110 will be transmitted to the target tissue T. As may be appreciated, to engage the tissue traction device 110 securely with the target tissue T, the distalmost end 110dm of the tissue traction device 110 (not necessarily visible in FIG. 5A, but illustrated in FIG. 1, FIG. 2, and FIG. 3) may be distally advanced into the target tissue T to penetrate the tissue, and the flexible elongate member 124 of the delivery device 120 may be rotated to rotationally advance the distal tissue-engagement zone 110e into the target tissue T to securely engage the tissue traction device 110 with the target tissue T. The delivery head 122 of the delivery device 120 may then be removed from its position within the space defined within the helical traction zone 110t of the tissue traction device 110, such as illustrated in FIG. 5B.

If the delivery head 122 of the delivery device 120 is in the form of a grasper, such as described above with reference to FIG. 2, and as illustrated in FIG. 5B, the delivery head 122, once removed from within the internal space of the traction zone 110t, may be used to engage the additional zone 110a of the tissue traction device 110 with a traction-adjusting site TA. As illustrated in FIG. 5B, the delivery head 122 is moved out of its initial delivery position as illustrated in FIG. 5A and operably engaged with the additional zone 110a to move the additional zone 110a to a traction-adjusting site TA. The configuration of the jaws 126 of the delivery head 122 may be shifted/actuated to grasp the additional zone 110a as well as the traction-adjusting site TA, such as in the deployed configuration of the tissue traction device 110 illustrated in FIG. 5C. In the illustrated example of an embodiment, the delivery head 122 includes at least first and second jaws 126 which together grasp the additional zone 110a and may be moved apart, with a portion of the additional zone 110a remaining therebetween, to further grasp tissue at the traction-adjusting site TA to engage the additional zone 110a with the tissue at the traction-adjusting site TA. As may be appreciated, the jaws 126 may be formed and configured and operably coupled and controlled in any of a variety of manners known to those of ordinary skill in the art, the present disclosure not being limited in this regard.

It will be appreciated that if the delivery head 122 of the delivery device 120 is inserted into the internal space defined by a generally helical-coil-spring-shaped traction zone 110t of a tissue traction device 110, such as in the examples of embodiments illustrated in FIGS. 1-4, rotation of the flexible elongate member 124 and/or the delivery head 122 may not effectively cause rotation of the distal tissue-engagement zone 110e to be distally advanced into and securely engaged with respect to the target tissue T. One solution to such challenge would be to form at least one non-circular/substantially flat/straight region or surface along one or both of the tissue traction device 110 or the delivery device 120. For instance, a region or surface along at least one of the zones of a tissue traction device formed in accordance with various principles of the present disclosure may be non-circular/substantially flat/straight to facilitate transmission of torquing of a delivery device formed in accordance with various principles of the present disclosure to rotation of the tissue traction device, and consequent advancement of the tissue traction device into the target tissue for secure engagement therewith.

An example of an embodiment of a tissue traction system 200 with a tissue traction device 310 with at least one flat section configured to engage at least one flat section of an associated delivery device 220 is illustrated in FIG. 6. Although the traction zone 310t defines a coiled traction zone 310t with square-shaped turns, a tissue traction device and delivery device of the present disclosure may include a variety of other structures shaped and configured to engage each other such that rotation of the delivery device is transmitted into rotation of the tissue traction device.

Returning to the example of an embodiment of a helical coil shaped tissue traction device 110 illustrated in FIGS. 1-3, a limited portion of the tissue traction device 110 may be formed to engage or be engaged with a component of a delivery device 120 such that rotation of the delivery device 120 is imparted to the tissue traction device 110. More particularly, in the illustrated example of an embodiment, a portion of the wireform at the transition between the distal tissue-engagement zone 110e and the traction zone 110t of the tissue traction device 110 is formed into a transition or cross-bar 110c positioned to be engaged by the delivery device 120 without rotating with respect to the delivery device 120. For instance, in the example of an embodiment illustrated in FIG. 2, the cross-bar 110c extends across the cross-section of the tissue traction device 110, e.g., diametrically across, or along a chord of the generally circular cross-section of the tissue traction device 110. It will be appreciated that the cross-bar 110c may, but does not necessarily, extend across an end of the traction zone 110t adjacent the distal tissue-engagement zone 110e. In some aspects, the cross-bar 110c is axially positioned along the tissue traction device 110 between the traction zone 110t and the distal tissue-engagement zone 110e. In some aspects, the cross-bar 110c extends across the interior area of the tissue traction device 110, such as to traverse the space in which the delivery head 122 of the delivery device 120 may be inserted, or is at least radially aligned with a space traversing the space in which delivery head 122 may be inserted. As such, a delivery head 122 in the form of a grasper device, such as illustrated in FIG. 2, may grasp the cross-bar 110c (e.g., the cross-bar 110c may be grasped by or between the jaws 126 of a delivery head 122 in the form of a clip). It will be appreciated that the illustrated cross-bar 110c is merely one example of a structure configured to impart rotational movement of delivery device to a tissue traction device, and other locations and configurations of rotational engagement structures imparting rotational movement between a tissue traction device and delivery device of the present disclosure are within the scope of the present disclosure.

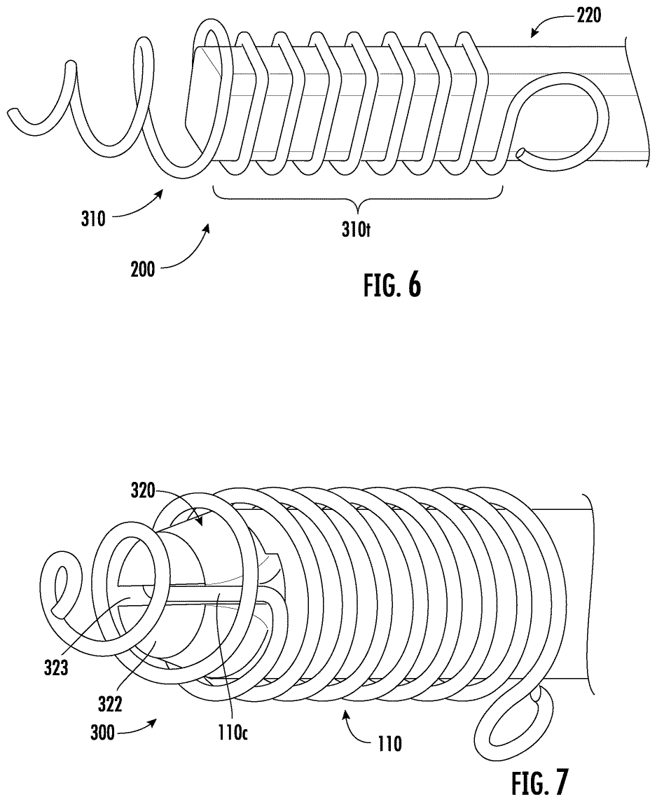

For instance, the example of an embodiment of a tissue traction system 300 illustrated in FIG. 7 has a simplified delivery device 320 which may be used with a tissue traction device 110 such as illustrated in FIGS. 1-3. The delivery device 320 is simplified (in comparison with the delivery device 120 of the tissue traction system 100 of FIG. 1 and FIG. 2) in that the delivery head 322 does not include moving parts such as grasper elements (e.g., jaws) which may engage a rotational engagement structure of the tissue traction device 110, such as the cross-bar 110c. Instead, the illustrated example of an embodiment of a delivery head 322 includes a non-circular/flat drive section positioned and configured to engage the cross-bar 110c of the tissue traction device 110. More particularly, in the illustrated example of an embodiment, the delivery head 322 includes a slot 323 sized, shaped, configured, and/or dimensioned to engage the cross-bar 110c of the tissue traction device 110. The shapes and/or configurations and/or positions of the slot 323 and the cross-bar 110c allow rotation of the delivery device 320 to rotate the tissue traction device 110, such as to advance the tissue traction device 110 into tissue.

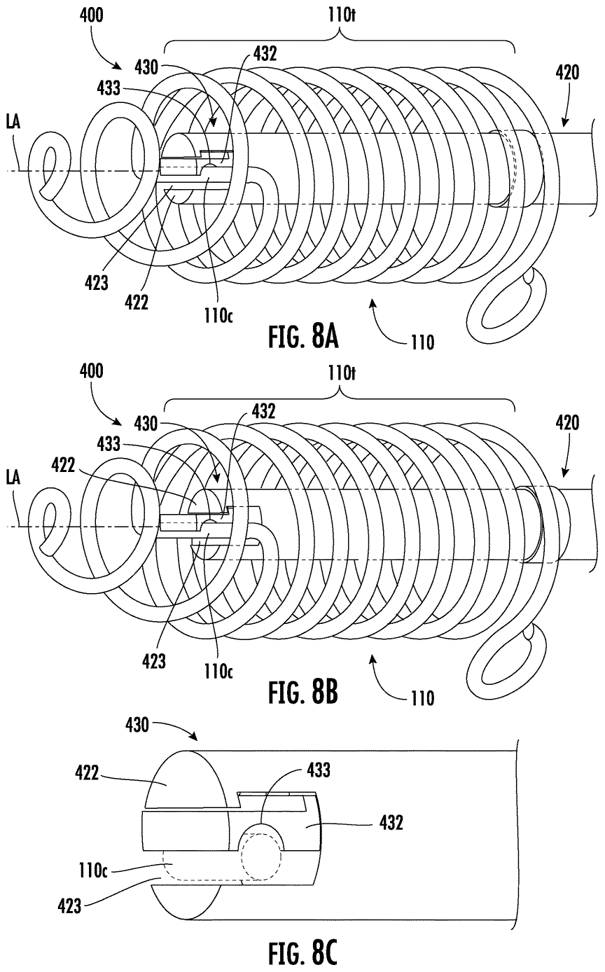

It will be appreciated that it may be beneficial for a tissue traction device and a delivery device of a tissue traction system 300 such as illustrated in FIG. 7 to be retained in axial engagement such that the delivery device does not inadvertently separate from the delivery device during delivery (e.g., during passage through tortuous pathways, and/or if the delivery device is retracted prior to deployment of the tissue traction device). In the example of an embodiment of a tissue traction system 400 illustrated in FIG. 8A and FIG. 8B, the delivery head 422 of the delivery device 420 a includes a slot 423 sized, shaped, configured, dimensioned, and positioned to receive the cross-bar 110c of the tissue traction device 110 as well as to retain the cross-bar 110c therein. More particularly, the delivery device 420 of the tissue traction system 400 of FIG. 8A and FIG. 8B includes a latch mechanism 430, illustrated in isolation in FIG. 8C, with a latch lock 432 movably positioned (e.g., axially translatable) within the slot 423 of the delivery head 422 to retain the cross-bar 110c of the tissue traction device 110 within the slot 423 of the delivery head 422. In the example of an embodiment illustrated in FIG. 8C, the latch lock 432 retains the cross-bar 110c within a retention slot 433 formed in the latch lock 432. In some aspects, the retention slot 433 is an undercut or other type of recess or channel in a surface of the latch lock 432 facing the cross-bar 110c, and generally extending generally parallel to the cross-bar 110c and optionally shaped to correspond to / complement the outer shape of the cross-bar 110c. The latch lock 432 is movable (e.g., axially translatable) with respect to the delivery head 422 and slot 423, such as by actuating an actuation mechanism (e.g., moving a control wire operably coupled to the latch lock 432 in a manner familiar to those of ordinary skill in the art) to release the cross-bar 110c from the slot 423. For instance, when the latch lock 432 of the example of an embodiment illustrated in FIGS. 8A-8C is advanced distally, the cross-bar 110c is no longer held within the slot 423 of the delivery head 422 and can be released from the latch lock 432 and the retention slot 433. The size, shape, configuration, and/or dimensions of the retention slot 433 can be made as small as possible to maintain the cross-bar 110c retained within the slot 423 when the latch mechanism 430 is in a locked configuration, yet to enable easy separation of the tissue traction device 110 from the delivery device 420 when the latch mechanism 430 is in the unlocked configuration. The relative dimensions of the traction zone 110t of the tissue traction device 110 and the latch mechanism 430 (e.g., in a direction transverse to the longitudinal axis of the tissue traction device 110, such as in a radial direction) may be selected to allow the cross-bar 110c to easily be released from any undercut formed by the retention slot 433. to allow the cross-bar 110c to move laterally out of the retention slot 433 to release the tissue traction device 110 from the delivery device 420, such as illustrated in FIG. 8B.

As noted above, a tissue traction device formed in accordance with various principles of the present disclosure may be provided with a rotational engagement structure other than a cross-bar defined between zones of a tissue traction device. For instance, whereas the tissue traction devices 110, 210 described thus far may be formed from a common, monolithic elongate element, with a cross-bar integrally formed by the common monolithic elongate element (e.g., between zones of the tissue traction device), a tissue traction device may be formed in accordance with various principles of the present disclosure as a multi-component tissue traction device formed from more than one component coupled with a coupler. In some aspects, the coupler includes a rotational engagement structure sized, shaped, configured, dimensioned, and/or positioned to be engaged with a delivery device, such as in a manner restricting (e.g., preventing) rotation between the coupler and the delivery device, to allow the delivery device to rotate the tissue-engagement zone of the tissue traction device and advance the tissue-engagement zone into tissue.

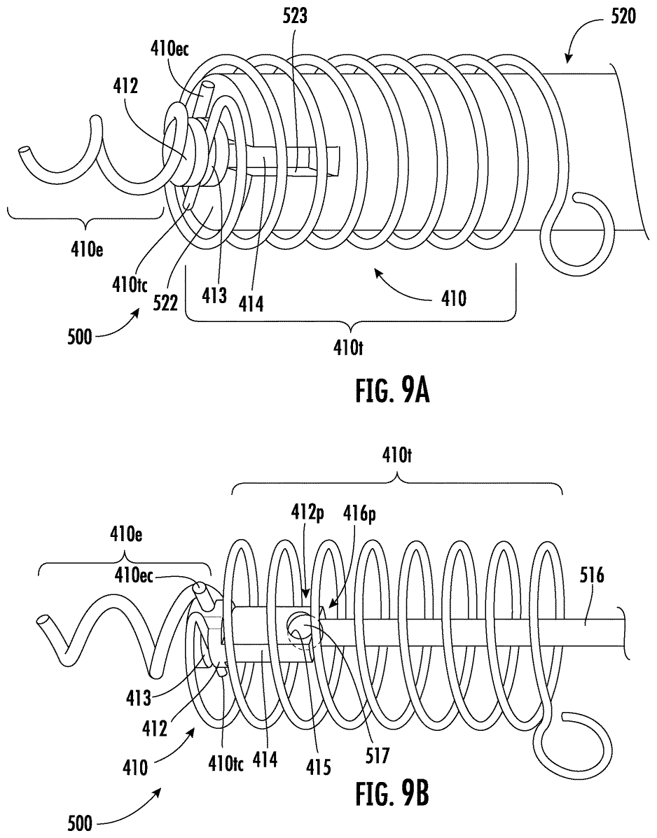

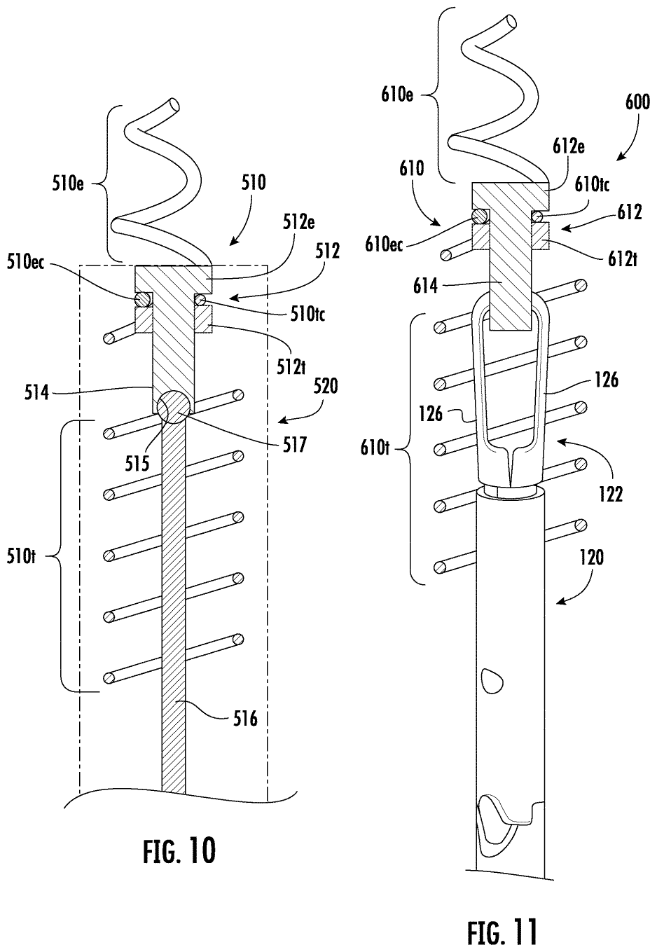

For instance, in the example of an embodiment of a tissue traction device 410 illustrated in FIG. 9A and FIG. 9B, a distal tissue-engagement zone 410e and a separately formed traction zone 410t of the tissue traction device 410 are operably coupled together with a coupler 412. In some aspects, the distal tissue-engagement zone 410e includes a cross-bar 410ec operably coupled to the coupler 412, and the traction zone 410t similarly includes a cross-bar 410tc operably coupled to the coupler 412 so that the distal tissue-engagement zone 410e and the traction zone 410t are thereby coupled to each other via the coupler 412. The coupler 412 may be overmolded, crimped, interference-fitted, or otherwise secured (e.g., fixedly coupled together) with the coupled ends of the distal tissue-engagement zone 410e and the traction zone 410t of the tissue traction device 410. For instance, in the illustrated example of an embodiment, the cross-bars 410ec, 410tc are interference-fitted within retainer slots of the coupler 412. Alternatively, as illustrated in FIG. 10 (partially in cross-section), a two-part coupler 512 may be used to couple together a distal tissue-engagement zone 510e and a traction zone 510t of the illustrated example of an embodiment of a tissue traction device 510. More particularly, a cross-bar 510ec of the distal tissue-engagement zone 510e and a cross-bar 510tc of the traction zone 510t may be held, trapped, retained, etc., between a first part 512e and a second part 512t of the coupler 512, with the parts 512e, 512t secured together in any of a variety of manners known to those of ordinary skill in the art (e.g., as a mechanical screw, a snap fit, adhesive, welding, etc.). In some aspects, the cross-bar 510ec of the distal tissue-engagement zone 510e is held, trapped, retained, etc., by the first part 512e of the coupler 512 (e.g., the first part 512e is overmolded, crimped, interference-fitted, or otherwise secured over the cross-bar 510ec), and/or the cross-bar 510tc of the traction zone 510t is held, trapped, retained, etc., by the second part 512t of the coupler 512 (e.g., the second part 512t is overmolded, crimped, interference-fitted, or otherwise secured over the cross-bar 510et), and the first part 512e and the second part 512t are secured together in any of a variety of manners known to those of ordinary skill in the art (e.g., a mechanical screw, a snap fit, adhesive, welding, etc.). The coupler 412 of the tissue traction device 410 of FIG. 9A and FIG. 9B, and the coupler 512 of the tissue traction device 510 of FIG. 10 are each configured to include one or more rotational engagement structures sized, shaped, configured, dimensioned, and/or positioned to engage the delivery device 520 of the example of an embodiment of a tissue traction system 500 illustrated in FIG. 9A (usable with either tissue traction device 410, 510) so that rotational movement of the delivery device 520 causes rotation of the tissue traction device 410, 510 engaged therewith. For instance, the illustrated examples of embodiments of couplers 412, 512 respectively include a proximally-extending extension 414, 514 with one or more non-circular (e.g., flat) surfaces engageable with corresponding surfaces of a delivery device 520. In the example of an embodiment of a tissue traction system 500 illustrated in FIG. 9A, the delivery device 520 is tubular to accommodate a coupler 412, 512 therein, and includes a rotational engagement structure in the form of a slot 523 formed in a delivery head 522 of the delivery device 520. The slot 523 is configured to engage the proximally-extending extension 414, 514 of the coupler 412, 512 such that rotation of the delivery device 520 (and thus the delivery head 522 with its slot 523) imparts rotation to the tissue traction device 410, 510 via the coupler 412, 512.

To retain the tissue traction device 410, 510 with respect to the delivery device 520, an elongate element 416, 516 may extend proximally from the proximally-extending extension 414, 514 to hold the tissue traction device 410, 510 proximally with respect to the delivery device 520. The elongate element 416, 516 may be releasably coupled to the proximally-extending extension 414, 514 to facilitate separation of the tissue traction device 410, 510 from the delivery device 520. In the examples of embodiments illustrated in FIG. 9B and FIG. 10, the releasable connection between the elongate element 416, 516 and the proximally-extending extension 414, 514 of the respective coupler 412, 512 is in the form of a ball and socket coupler, such as known to those of ordinary skill in the art. More particularly, the proximal end 412p, 512b of the respective coupler 412, 512 (e.g., the proximal end of the proximally-extending extension 414, 514) defines a socket 415, 515, respectively, sized, shaped, configured, and/or dimensioned to securely receive a respective ball component 417, 517 provided on the distal end 416d, 516d of the respective elongate element 416, 516. The engagement of the ball component 417, 517 within the respective socket 415, 515 is sufficiently secure to withstand forces which may be encountered while advancing or retracting the tissue traction system 500 within a patient's body, to retain the tissue traction device 410, 510 engaged with the delivery device 520. To separate and deploy the tissue traction device 410, 510 from the delivery device 520, a medical professional must proximally pull on the elongate element 416, 516 to overcome the retention force between the ball component 417, 517 and the respective socket 415, 515. Advantageously, the delivery head 522 may be held against the coupler 412, 512 while a proximal force is exerted on the elongate element 416, 516 so that the proximal force is not transmitted to the tissue traction device 10, 510 (which may effect undesirable retraction of the tissue traction device 410, 510 from its deployed position engaging target tissue).

It will be appreciated that a coupler between components of a tissue traction device formed in accordance with various principles of the present disclosure, such as described with reference to the examples of embodiments illustrated in FIG. 9A, FIG. 9B, and FIG. 10, may be engaged by a grasper-type delivery head of a delivery device, such as illustrated in FIG. 1 and FIG. 2. For instance, the example of an embodiment of a tissue traction system 600 illustrated in FIG. 11 includes a tissue traction device 610 with a coupler 612 which is configured to couple together a distal tissue-engagement zone 610e and a traction zone 610t of the tissue traction device 610, as well as to be engaged by jaws 126 of a delivery head 122 of a delivery device 120 such as illustrated in FIG. 1 and FIG. 2. More particularly, the illustrated example of an embodiment of a coupler 612 is similar to the example of an embodiment of a coupler 512 illustrated in FIG. 10, but includes a proximally-extending extension 614 which is sized, shaped, configured, and/or dimensioned to be grasped by the jaws 126 of the delivery head 122. It will be appreciated that the coupler 612 may, instead, be similar to the coupler 412 illustrated in FIG. 9A and FIG. 9B, except for an increased axial extent of the proximally-extending extension 614 for being grasped by the jaws 126 of the delivery head 122 of the delivery device 120. Various features of the coupler 612 and its operably association with the tissue-engagement zone 610e and/or the traction zone 610t of the tissue traction device 600 (e.g., parts 612e, 612t) may be substantially the same as described above with reference to the coupler 412 or the coupler 512, reference being made to the above descriptions, for the sake of brevity and without intent to limit, as applicable mutatis mutandis to the coupler 612 illustrated in FIG. 11. In some aspects, the cross-bar 610ec of the distal tissue-engagement zone 610e, and/or the cross-bar 610et of the traction zone 610t are held, trapped, retained, etc., between the first part 612e of the coupler 612 and the second part 612t of the coupler 612, such as being sandwiched therebetween without necessarily being individually held, trapped, retained, etc., independently in a part 612e, 612t of the coupler 612. For instance, a cross-bar 610ec, 610tc may be held in an open channel of one of the parts 612e, 612t of the coupler 612 facing the other of the part 612e, 612t of the coupler 612. The first part 612e and the second part 612t may then be secured together in any of a variety of manners known to those of ordinary skill in the art (e.g., a mechanical screw, a snap fit, adhesive, welding, etc.) to retain the cross-bars 610ec, 610tc therebetween. It will be appreciated that a similar arrangement may be used in forming the coupler 512 of the example of an embodiment illustrated in FIG. 10.

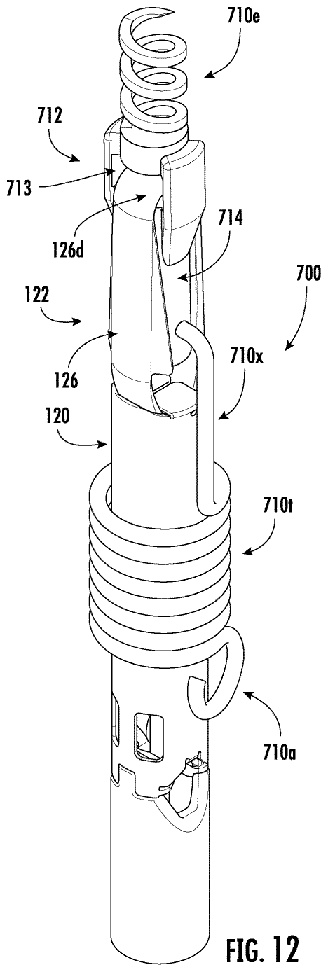

Another example of an embodiment of a tissue traction system 700 with a multi-component tissue traction device 710 is illustrated in FIG. 12. The distal tissue-engagement zone 710e and the traction zone 710t of the tissue traction device 710 of FIG. 12 are operably coupled via a coupler 712. The coupler 712 extends proximally from the distal tissue-engagement zone 710e for engagement by a leg or extensions 710x of the traction zone 110t of the tissue traction device 110. Similar to the examples of embodiments of multi-component tissue traction devices 410, 510, 610 illustrated in FIG. 9A, FIG. 9B, FIG. 10, and FIG. 11 and discussed above, a component of the multi-component tissue traction device 710 illustrated in FIG. 12 is grasped by a grasper-type delivery device 120. In the illustrated example of an embodiment, the grasper-type delivery device 120 is similar to the example of an embodiment of a delivery device 120 illustrated in FIG. 1, FIG. 2, FIG. 5A, and FIG. 5B, with a delivery head 122 including at least two jaws 126. The example of an embodiment of a coupler 712 of the tissue traction device 710 illustrated in FIG. 12 is configured to accommodate and to be engaged by the jaws 126 of the tissue traction device 110 in a manner in which rotation of the delivery device 120 causes rotation of the tissue traction device 710. More particularly, distal ends 126d of the jaws 126 are extendable into a slot 713 defined in a coupler 712 to impart movement of the delivery device 120, via the delivery head 122, to the distal tissue-engagement zone 710e as well as to retain the tissue traction device 710 with respect to the delivery device 120, at least during delivery. Once rotation of the delivery device 120 advances the distal tissue-engagement zone 710e into engagement with tissue, the jaws 126 of the delivery device 120 may be shifted from the illustrated substantially closed configuration to an open configuration to release and deploy the distal tissue-engagement zone 110e with respect to tissue. The traction zone 710t remains coupled to the tissue-engagement zone 710e and the additional zone 710a may be grasped (e.g., by the jaws 126 of the delivery head 122 of the delivery device 120) and coupled with a traction-adjusting site, such as described above with reference to FIG. 5C. As may be appreciated, a tissue traction system of the present disclosure provides a tissue traction device which may be engaged directly into target tissue without the need of a separate tissue-engagement element to couple the tissue traction device to the target tissue. As such, the delivery and deployment of a tissue traction device is simplified, with fewer instruments and steps than required by prior art devices and systems and method. Optionally, the tissue traction system, once deployed, may utilize a grasper feature of the delivery device used to deliver and engage the tissue traction device with tissue to engage an opposite end of the tissue traction device with a traction-adjusting site spaced apart from the target tissue to apply traction to the target tissue. As such, a tissue traction system formed in accordance with various principles of the present disclosure reduces the number of instruments and/or components which require manipulation by the medical professional (in addition to the instruments which must be manipulated to perform the procedure) and which may occupy the limited anatomical area at which the procedure is being performed. In some aspects, the helical form of a tissue-engagement element of the tissue traction device may be unscrewed to rotationally withdraw from the tissue, and then may be repositioned at another tissue site.

It will be appreciated that devices and systems of the present disclosure may be used for treating various anatomical tissue and/or anatomical systems, such as gastrointestinal, abdominal, digestive, urinary, reproductive, respiratory, cardiovascular, circulatory, etc. It will be appreciated that reference to a body passage includes naturally-existing passages (e.g., the colon) as well as medically-created passages (e.g., a passage created with the use of a medical instrument, and not existing without medical intervention) or otherwise. It will further be appreciated that the medical devices and systems described herein may be navigated within and through various body lumens, or other lumens, including, for example, catheters, ureteroscopes, bronchoscopes, colonoscopes, duodenoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. In particular, it will be clear to those skilled in the art that principles of the present disclosure may be embodied in other forms, structures, arrangements, proportions, and with other elements, materials, and components, without departing from the concept, spirit, or scope, or characteristics thereof. Other examples of manners of implementing the disclosed principles will occur to a person of ordinary skill in the art upon reading this disclosure. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations, and that the various separate features of the present subject matter need not all be present in order to achieve at least some of the desired characteristics and/or benefits of the present subject matter or such individual features. Moreover, elements shown as integrally formed may be constructed of multiple parts or elements shown as multiple parts may be integrally formed, the operation of elements may be reversed or otherwise varied, the size or dimensions of the elements may be varied. It should be apparent to those of ordinary skill in the art that variations and/or modifications, such as of structure, arrangement, proportions, materials, components, and otherwise, can be applied to the disclosed devices, systems, and/or methods, and/or to the sequence of steps of the method described herein without departing from the concept, spirit, and scope of the disclosure. For instance, variations or modifications may be made to adapt devices, systems, or methods of the present disclosure to specific environments and operative requirements without departing from the principles or spirit or scope of the present disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes, modifications, and/or additions apparent to those skilled in the art are deemed to be within the spirit, scope, and concept of the disclosure as defined by the appended claims. Various further benefits of the various aspects, features, components, and structures of tissue traction devices, systems, and methods such as described above, in addition to those discussed above, may be appreciated by those of ordinary skill in the art. The foregoing discussion has broad application and has been presented for purposes of illustration and description and is not intended to limit the disclosure to the form or forms disclosed herein.

The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A traction device for applying traction to anatomical tissue within a patient, said traction device comprising:

2. a traction zone configured to impart traction to the anatomical tissue; and

3. a tissue-engagement zone operably coupled with the traction zone;

4. wherein the tissue-engagement zone is in the form of a helical coil.

5. The traction device of claim 1, wherein said traction zone is in the form of a helical coil spring.

6. The traction device of claim 2, wherein the traction zone and the tissue-engagement zone are formed from the same monolithic elongate element.

7. The traction device of claim 2, wherein the traction zone and the tissue-engagement zone are formed separately from each other and are coupled together via a coupler.

8. The traction device of claim 1, wherein the distalmost end of said tissue-engagement zone is sharp and pointed to penetrate target tissue to which said traction device is to apply traction.

9. The traction device of claim 1, further comprising an additional zone operably coupled to said traction zone and configured to be attached to tissue spaced apart from target tissue to which said tissue-engagement zone is to be attached.

10. The traction device of claim 6, wherein said additional zone is configured to be grasped by a tissue-engagement element configured to grasp the additional zone and the tissue spaced apart from the target tissue.

11. The traction device of claim 6, wherein said additional zone is in the form of a helical coil.

12. A tissue traction system for applying traction to anatomical tissue within a patient, said tissue traction system comprising:

13. a tissue traction device defining a tissue-engagement zone in the form of a helical coil, and a traction zone extending proximally from said tissue-engagement zone; and

14. a delivery device having a delivery head configured to engage said tissue traction device and to rotate said delivery device to rotationally advance said tissue-engagement zone into anatomical tissue.

15. The tissue traction system of claim 9, wherein said tissue traction device defines a cross-bar extending across the cross-section of said tissue traction device and configured to be rotationally engaged by said delivery head of said delivery device.

16. The tissue traction system of claim 10, wherein said delivery head comprises a pair of jaws positionable with respect to said tissue traction device to grasp said cross-bar of said tissue traction device.

17. The tissue traction system of claim 10, wherein said delivery head defines a slot for receiving said cross-bar of said tissue traction device.

18. The tissue traction system of claim 9, wherein said tissue traction device comprises a coupler joining said tissue-engagement zone and said traction zone of said, said coupler configured to be rotationally engaged by said delivery head of said delivery device.

19. The tissue traction system of claim 13, wherein said delivery head comprises a pair of jaws movable towards each other to grasp a portion of said coupler to impart rotational movement to said tissue traction device.

20. The tissue traction system of claim 13, wherein said delivery head defines a rotational engagement structure configured to engage a rotational engagement structure of said coupler to impart rotational movement to said tissue traction device.

21. The tissue traction system of claim 9, wherein said delivery head comprises a pair of jaws movable towards each other to grasp a portion of said tissue traction device to impart rotational movement to said tissue traction device.

22. The tissue traction system of claim 16, wherein said tissue traction device further comprises an additional zone extending proximally from said traction zone.

23. The tissue traction system of claim 17, wherein said jaws are positionable within said traction zone to deliver said tissue traction device to target tissue within the patient, and are removable from within the traction zone to grasp said additional zone and anatomical tissue.

24. A method of applying traction to anatomical tissue, said method comprising:

25. engaging a distal end of a helical tissue-engagement zone of a tissue traction device with target tissue along the anatomical tissue;

26. rotating the helical tissue-engagement zone to advance the tissue-engagement zone into the anatomical tissue; and

27. coupling a traction zone of the tissue traction device, proximally extending from the helical tissue-engagement zone, to anatomical tissue at a traction-adjusting site spaced apart from the target tissue to apply traction to the target tissue.

28. The method of claim 19, wherein the traction zone is in the form of a helical coil, and rotating the helical tissue-engagement zone comprises rotating a delivery head of a delivery device within the helical traction zone to cause the helical tissue-engagement zone to rotate.
